(19) United States
(12) Patent Application Publication
(10) Pub. No.: US 2012/0034308 A1
(43) Pub. Date: Feb. 9, 2012

(54) REDUCED-CHOLESTEROL DAIRY PRODUCT FOR USE AS A MEDICAMENT

(75) Inventor: Daniel Dalemans, Herstal (BE)

(73) Assignee: S.A. Corman, Goe (BE)

(21) Appl. No.: 13/262,606

(22) PCT Filed: Apr. 23, 2010
(86) PCT No.: PCT/EP2010/055424

§ 371 Date: Sep. 30, 2011

(30) Foreign Application Priority Data
Apr. 23, 2009 (EP) 09158666.9

(51) Int. Cl. A61K 35/20; A61P 3/06; A61P 1/16; A61P 25/00; A61P 9/00; A61P 29/00

(52) U.S. Cl. 424/535

(57) ABSTRACT

A ruminant reduced-cholesterol dairy product is used as a medicament.

### FIELD OF THE INVENTION

The present invention is in the field of therapy and/or prevention of mammal (especially human) diseases and is related to a dairy product (milk or milk product) including food or feed compositions comprising this dairy product, this dairy product being obtained from a ruminant, preferably a cattle dairy product (milk or milk product), more preferably a cow dairy product (milk or milk product) with a reduced cholesterol content (for its use) as a medicament.

### BACKGROUND OF THE INVENTION

There is a tendency in developed countries to consume food rich in triglycerides or fat (i.e. about 22% of total intake; measured on dry weight) and in cholesterol.

Lipid disorder is a medical term to define high blood cholesterol and triglycerides mammal blood ratios, which is linked to the risk increase of cardiovascular (such as atherosclerosis) and other heart diseases in mammals, especially in humans.

Milk contains about 3 to about 8% of fat (w:w), that is mainly a saturated fat.

Fat (triglycerides) is essential in milk for its flavor and for some of its ‘technical’ properties, especially in milk-derived products (dairy products), such as spreads, cheeses, creams, and so on.

Fat content in milk-derived product may be of about 3% to about 100% of dry weight (between about 10% and 80% on total weight). For instance cheese and butter contains about 45% (expressed in dry weight) and about 80% of fat (expressed in total weight or up to 98% in dry weight), respectively.

Unsaturated fatty acids, especially ω3 poly-unsaturated fatty acids, such as α-linolenic acid (ALA), Eicosapentaenoic acid (EPA) and/or Docosahexaenoic acid (DHA) appear to exert positive health effects and their properties are presented in functional food compositions, food complements or nutraceuticals enriched in these poly-unsaturated fats.

These specific fatty acids may be present in the form of fat (triglycerides), phospholipids, or as fatty acids conjugated to carriers, such as lipoproteins.

However, (ruminant) milk is poor on (poly-) unsaturated fatty acids. For regulatory reasons, a dairy product (milk product) enriched with exogenous fatty acids (for instance by adding vegetal fat) may not be labeled as a dairy product (milk-product).

Milk fat further contains cholesterol (about 300 mg for 100 g of fat). However, cholesterol food intake should be limited to a maximum of 300 mg per day. Consequently, improving the nutritional image of milk fat involves the reduction of its cholesterol content.

Both cholesterol and saturated fat are known to adversely affect (human) health, especially with regard to cardiovascular diseases and to some cancers.

Therefore, whole milk, milk fat and dairy products, which are rich in this milk fat (such as spreads, creams, cheeses . . . ) and any food or feed compositions comprising these dairy products suffer from a bad nutritional image, because of both their high saturated fatty acids and their high cholesterol contents linked to these health issues.

### STATE OF THE ART

Presently, extraction of cholesterol from milk fat is achieved industrially by physical methods, such as molecular encapsulation by contact with a cyclodextrin (EP-387708 B1) or by steam stripping. With these methods, a minimum of 75% of the initially present cholesterol may be extracted.

Alternatively, several methods were developed to reduce cholesterol content in milk, or its bio-availability.

For instance U.S. Pat. No. 6,485,931 B2 describes milk-cholesterol conversion into coprostanol, with a lower availability.

The international patent application WO 2004 052 122 describes methods to reduce the cholesterol content of animal-derived fat by changing their regimen.

The European patent EP 1585508 B1 is related to the use of sphingolipids for the manufacture of a food or pharmaceutical product for lowering cholesterol and triglyceride in plasma and/or serum and to the use of sphingolipids for the treatment or prevention of cardiovascular diseases in humans through lowering cholesterol and triglyceride in plasma and/or serum.

EP 0615 690 describes the extraction of milk-cholesterol using oils.

U.S. Pat. No. 5,175,015 describes a skimmed milk mixed with vegetable fat.

### AIMS OF THE INVENTION

The present invention aims to provide a food or feed element or a food or feed composition that does not present the drawbacks of the state of the art, especially an element or composition that may prevent and/or treat (or improve) cardiovascular mammals (including human) diseases and/or related diseases, such as cancer and in general to improve the health of these mammals, especially of humans.

The present invention aims to provide such element or composition that can be used as a functional food or feed (composition) or as a nutraceutical and that can be presented in usual dairy products or in feed or food compositions comprising these dairy products (milk and milk beverages, spreads, especially butter, creams, cheese, etc) or obtained from these dairy products (ice creams, bakery and confectionery products, etc)

### SUMMARY OF THE INVENTION

The present invention relates also to this ruminant milk (fat), preferably a cow milk (fat) as a medicament.

The dairy product (milk or milk product), especially the dairy fat, of the invention has a low cholesterol content, possibly no (detectable level of) cholesterol, preferably comprised between about 10 mg/100 g fat and about 150 mg/100 g fat, preferably comprised between about 30 mg/100 g fat and about 150 mg/100 g fat, more preferably comprised between about 30 mg/100 fat and about 90 mg/100 g fat.

Preferably the dairy product (milk or milk product), especially the dairy fat, of the invention is obtained from a ruminant, more preferably obtained from a cow.

Possibly the dairy product (milk or milk product), especially the dairy (milk) fat of the invention contains from about 60% to about 70% of saturated fat (w_{saturated fat}:W_{total fat}).

Advantageously, the dairy product of the invention is a milk that comprises between about 3% and about 4% (w:w) of protein (of which about 80% of casein), between about 3% and about 6% (w:w) of fat, between about 4% and about 5% (w:w) of carbohydrates and between about 0.6 and about 1% of minerals, being mainly calcium, potassium, chloride and phosphorus (i.e. compared to the total weight % of the dairy product being 100%).

Alternatively, the dairy product of the invention is a dairy product enriched in milk fat, such as a spread, preferably a butter, a milk beverage, a cream or a cheese. The present invention is also related to a (functional) food or feed composition comprising the dairy product or the dairy fat of the invention or obtained from the dairy product or dairy fat of the invention, such as ice cream, bakery or confectionary products.

By ‘about’, it is preferably meant every real number plus or minus 10%. For example by ‘about 4%’, it is meant every real numbers between 3.6% and 4.4%.

The ruminant dairy product (milk, milk product or fat) of the present invention and obtained from the ruminant milk, may be used alone (as milk, a milk cream or anhydrous milk fat), as a derived product (as a spread like butter, a milk beverage or cheese), or as a supplement or active ingredient in a (functional) feed composition or in a (functional) food composition with the other usual ingredients of this composition, as or in a nutraceutical composition, and/or as or in a pharmaceutical composition.

Advantageously, this medicament (pharmaceutical composition) and/or nutraceutical comprising the dairy product, especially the dairy fat, of the invention is provided (present) in a food composition, preferably a spread, more preferably a butter having between about 10% and about 50% (preferably of about 40%) (w:w) total fat (lipid) and a cholesterol content comprised between about 10 mg/100 g fat and about 150 mg/100 g fat, preferably between about 30 mg/100 g fat and about 150 mg/100 g fat, more preferably between about 30 mg/100 fat and about 90 mg/100 g fat.

The consumption of the dairy product, especially the dairy fat, of the present invention advantageously increases (long chain) poly-unsaturated fatty acids content (such as Arachidonic acid, Eicosapentaenoic acid and Docosahexaenoic acid) in a mammal (including the human) blood.

More precisely, the consumption of dairy product, especially the dairy fat, of the present invention further increases long chain poly-unsaturated ω3 fatty acids content in a mammal (including the human) blood.

By long chain ω3 fatty acids, it is meant Eicosapentaenoic acid (EPA) and/or Docosahexaenoic acid (DHA).

The consumption of the dairy product, especially the dairy fat, of the invention further decreases the triglyceride content in a mammal (including the human) blood (compared to a ruminant milk fat that does not have a reduced cholesterol content (having a cholesterol content of about 280 mg/100 g fat)).

The consumption of the dairy product, especially the dairy fat, of the invention increases by at least about 15% (preferably by at least about 30%, more preferably by at least about 50%) the HDL-cholesterol content in a mammal (including the human) blood and preferably increases non-HDL blood cholesterol content by less than about 10%, more preferably by less than about 20%.

Therefore, the consumption of the dairy product, especially the dairy fat, of the present invention also decreases the atherogenic ratio of mammal (including a human) blood by at least about 10%, preferably by at least about 20%, more preferably by at least about 30%.

The present invention further relates to a food (medicament and/or nutraceutical) composition enriched in the dairy product, especially the dairy fat, of the invention, this food having a low cholesterol content and comprising between about 12% and about 90% (preferably between about 22% and about 50%, more preferably between about 35% and about 45%) (w:w; on dry weight) fat (lipid), for decreasing the total cholesterol content of mammal (including the human) blood (compared to a ruminant milk fat not having a reduced cholesterol content (having a cholesterol content of about 280 mg/100 g fat)).

Advantageously, the food (medicament and/or nutraceutical) of the invention is a dairy product selected from the list consisting of spread, preferably butter and spreadable butter, ice cream, milk cream, cheese, fermented milk, flavored milk and cream.

Another aspect relates to a regimen whereby milk fat is replaced by reduced-cholesterol milk fat and/or regimen enriched in reduced-cholesterol milk-fat, preferably the reduced-cholesterol milk-fat of the invention, for use as a medicament.

A preferred regimen for use as a medicament contains from about 10% to about 22% of total fat (w:w; dry weight) including milk fat (having a reduced cholesterol content) and a reduced cholesterol content (comprised between about 10 mg/100 g (total) fat and about 150 mg/100 g (total) fat, preferably comprised between about 30 mg/100 g (total) fat and about 150 mg/100 g (total) fat, more preferably comprised between about 30 mg/100 (total) fat and about 90 mg/100 g (total) fat).

Advantageously, the dairy product (or regimen containing the dairy product of the invention) especially the dairy fat, of the invention is used in the treatment and/or the prevention of a neurological disease, preferably a disease selected from the group consisting of depression (including post-partum depression), Alzheimer's disease, behavioural disorders, such as Attention Deficit Hyperactivity Disorder (ADHD) and hyperactivity, as well as developmental coordination disorder, epilepsy or a mixture thereof.

The dairy product (or regimen containing the dairy product), especially the dairy fat, of the invention can be further used in the treatment and/or the prevention of an inflammatory disease, preferably selected from the group consisting of a Chronic inflammatory condition, such as rheumatoid arthritis, a skin disorder, a gastrointestinal disorder (Inflammatory Bowel Disease, Crohn's disease), an allergic sensitivity, especially in very young children, pneumonia, reduced lung/breathing capacity and chronic pulmonary disorder or a mixture thereof.

The dairy product (or regimen containing the dairy product) of the invention can be further used in the treatment and/or prevention of cardiovascular diseases and/or liver diseases.

Preferably, the dairy product (or regimen), especially the dairy fat, according to the present invention is for use in the treatment and/or the prevention of liver disease, being more preferably steatosis.

Preferably, the dairy product (or regimen), especially the dairy fat, according to the present invention is for use in the treatment and/or the prevention of a cardiovascular disease and/or of a liver disease and/or of an inflammatory disease and/or of a neurological disease by decreasing atherogenic ratio in mammal (including the human) blood by at least 10% (preferably by at least about 20%, still more preferably by at least about 30%) and/or by increasing (long chain) poly-unsaturated fatty acids content in mammal (including the human) blood and/or possibly by decreasing the triglyceride content in mammal (including the human) blood.

The present invention also relates to a ruminant dairy product with a low cholesterol content (milk or milk product with a low cholesterol content), especially a dairy fat, supplemented with an ω3 fatty acid source, preferably selected from the group consisting of α-linolenic acid (ALA), Eicosapentaenoic acid (EPA) and Docosahexaenoic acid (DHA) (wherein ω3 fatty acids are added at about 0.5% (w_{ω3}:w_{fat}) to about 10% (w_{ω3}:w_{fat}), preferably at about 2% (w_{ω3}:w_{fat}) to about 8% (w_{ω3}:w_{fat}), preferably at about 6% (w_{ω3}:w_{fat}) (i.e: compared to the total weight of the dairy product fat being 100%), possibly for use as a medicament.

More preferably, the present invention relates to a ruminant dairy product (milk or milk product), especially a dairy fat possibly supplemented (added with) with an ω3 fatty acid source being α-linolenic acid (ALA), (having a (molar) ratio of ALA to linoleic acid comprised between about 0.2 and about 1.5, preferably between about 1 and about 1.5) with a reduced cholesterol content), possibly for use as a medicament.

Another aspect of the present invention is related to a method to increase the content in a mammal (including the human) blood (or plasma) of long chain poly-unsaturated (ω3) fatty acid by a consumption of the dairy product (or regimen containing the dairy product), especially the dairy fat, of the invention, preferably in replacement of a (ruminant) milk fat not having a reduced cholesterol content (having a cholesterol content of about 280 mg/100 g fat).

Still another aspect of the present invention is related to a method to reduce triglyceride content in a mammal (including the human) blood by a consumption of the dairy product (or regimen containing the dairy product), especially the dairy fat, of the invention, preferably in replacement of a (ruminant) milk fat not having a reduced cholesterol content (having a cholesterol content of about 280 mg/100 g fat).

A further aspect of the invention is related to a method for increasing by at least about 15% (preferably by at least about 30%, more preferably by at least about 50%) the HDL-cholesterol content in mammal (including the human) blood and preferably by increasing non-HDL cholesterol content by less than about 20%, more preferably by less than about 10%, in this mammal (including the human) blood by a consumption of the dairy product (or regimen containing the dairy product), especially the dairy fat, of the invention.

Therefore, this consumption in the method of the invention provides a decrease of the mammal blood atherogenic ratio by at least about 10%, preferably by at least about 20%, still more preferably by at least about 30%.

A last aspect of the present invention is related to a method to reduce the cholesterol content in a mammal (including the human) blood by the consumption of a feed or food composition comprising (enriched with) or obtained from the dairy fat (or the dairy product of the invention), this feed or food composition having a lipid content comprised between about 3% (milk) and about 85% (butter), preferably of about 22% and about 50%, more preferably between about 35% and about 45%) (w:w; calculated on total weight) or between about 20% (milk) and about 98% (butter) (w:w calculated on dry weight), and a low cholesterol content instead of the (ruminant) milk fat not having a reduced cholesterol content (having a cholesterol content of about 280 mg/100 g fat).

### DETAILED DESCRIPTION OF THE INVENTION

As described in the method of the European patent EP-387708 B1, the inventors reduced the cholesterol content of cow milk by treating it with β-cyclodextrin.

More precisely, 5 kg cow milk fat containing 278 mg cholesterol/100 g fat was mixed during 30 minutes at 55° C. with 5 kg of a 6% beta-cyclodextrin solution to form a “oil in water” emulsion. Milk fat with a lower content of cholesterol (25 mg/100 g) was recovered by continuous centrifugation, washing with water, second continuous centrifugation and drying under vacuum.

The inventors treated 56 hamster animals, being a cardiovascular model adapted to the human population with several regimens that mimics human diets (regarding fat content of 12.5% and 22% (% on dry weight)) and containing milk fat treated or not and with a classical (milk fat free) regimen.

By comparing animals fed with regimen (heavily) enriched with milk fat and classical regimen, the inventors observed a consistent increase in blood cholesterol and a tendency of increase in blood triglycerides. However, atherogenic ratio, being the HDL/non-HDL cholesterol ratio was only marginally impaired.

Therefore, the inventors concluded that feeding an animal (including a human) with a cholesterol-rich milk fat (or untreated, i.e. containing about 280 mg cholesterol per 100 g fat) results in the worsening of most of blood parameters with regard to a cardiovascular risk and associated diseases, consistent with the bad nutricional image of dairy products.

Furthermore, the inventors observed that the liver of hamsters fed with the cholesterol-rich milk fat is bigger, as well as their cholesterol and triglyceride contents, which are increased by about 6-fold and by about 2-fold.

The inventors measured in parallel the same parameters for hamsters fed with the reduced cholesterol milk fat (25 mg/100 g) of the invention.

By comparison with hamsters fed with cholesterol-rich milk fat, the inventors measured a consistent reduction in blood cholesterol, in triglycerides and in atherogenic ratio in animals fed with the reduced cholesterol fat of the invention.

The inventors conclude therefore that (all) the major blood parameters relevant to a measure of a cardiovascular risk and associated diseases are improved by the dairy product of the invention (reduced-cholesterol milk fat) by comparison to animals fed with normal milk fat (cholesterol rich).

By comparison with hamsters fed with the classical regimen, blood triglyceride content is not consistently improved in animals fed with the reduced-cholesterol milk fat of the invention.

Interestingly, by feeding animals with reduced-cholesterol milk fat (instead of the classical regimen), the ‘bad’ (non-HDL) cholesterol did not increase, while the good (HDL) cholesterol consistently increased, especially when giving regimens (comprising the dairy fat of the invention) at 22% of lipids, that mimic current Human diets, especially in Europe, USA and Japan.

The liver parameters such as total weight and cholesterol content (of animals fed with the dairy fat of the invention) are similar to those of hamsters fed with classical regimen (not comprising milk fat), with the exception of triglyceride and phospholipid contents that were higher.

In all cases, the atherogenic ratio is consistently and significantly decreased in animals fed with the reduced-cholesterol milk fat of the invention.

The inventors therefore concluded that, although they fed animals with a product that is rich in saturated fat, their blood and liver parameters were not degraded and, in some aspects, they were improved.

Further refining their observations, the inventors measured a consistent and significant increase of EPA and DHA plasma contents in animals fed with the reduced-cholesterol milk of the invention.

Since there is a need to increase the blood content of long chain ω3 fatty acids in animals (including humans), the inventors deduce that the low-cholesterol milk fat they tested is a very suitable nutraceutical, functional feed (or food) composition or an adequate and efficient pharmaceutical ingredient or composition.

The inventors conclude that, in long terms, this reduced-cholesterol milk fat is safe and even improves several blood parameters of an mammal (including a human).

The inventors further fed other group of hamsters with milk fat enriched with ω3 essential fatty acid α-linolenic acid (ALA).

With the exception of a strong increase in plasma content of the α-linolenic acid, the inventors noticed only a marginal reduction of triglycerides and cholesterol contents in their blood by comparison to control groups.

The inventors further noticed that both the addition to the milk fat of the ω3 fatty acid α-linolenic acid and the reduction of the cholesterol content of this milk fat (enriched in ALA) synergize and provoke a strong reduction of the blood cholesterol and triglyceride contents, coupled with a consistent increase of the plasma content of the three ω3 fatty acids measured (ALA, EPA and DHA).

Therefore, although one may fear that an animal (including a human) fed with low-cholesterol milk (fat) will have increased blood and liver triglyceride values (and will be at higher cardiovascular risk), these comparative data clearly shows that this feared increase is not necessary spectacular or consistent and does not translate into an increase of the liver size. More importantly, the reduced-cholesterol milk fat of the invention results in an increase of long chain ω3 fatty acids, such as EPA and DHA measured in the mammal plasma, these long chain ω3 fatty acids being known to improve several health related aspects. The present invention will be described in more details in the following non limiting examples.

The inventors developed a regimen whereby milk fat is replaced by low-cholesterol milk fat and/or regimen enriched in low-cholesterol milk-fat.

A typical regimen contains from about 10 to about 22% of total fat (w:w; dry weight) and a reduced cholesterol content (comprised between about 10 mg/100 g (total) fat and about 150 mg/100 g (total) fat, preferably comprised between about 30 mg/100 g (total) fat and about 150 mg/100 g (total) fat, more preferably comprised between about 30 mg/100 (total) fat and about 90 mg/100 g (total) fat).

Alternatively, the skilled person and/or the consumer may replicate such a regimen by replacing dairy products usually taken (and possibly by reducing its consumption of other cholesterol-rich products such as meat) by the dairy products of the invention (that contain the reduced-cholesterol milk fat of the invention).

### EXAMPLES

### Example 1

The inventors divided 56 hamsters in 8 groups.
Group G1: hamsters fed with fat from winter milk.
Group G2: hamsters fed with fat from winter milk further depleted in cholesterol (i.e. low-cholesterol milk-fat).
Group G3: hamsters fed with fat from spring milk.
Group G4: hamsters fed with fat from spring milk further enriched in ALA.
Group G5: hamsters fed with fat from spring milk further strongly enriched in ALA.
Group G6: hamsters fed with fat from spring milk further depleted in cholesterol and enriched in ALA.
Group G7: hamsters fed with fat from spring milk further depleted in cholesterol and strongly enriched in ALA.
Group G8: control hamsters having received classical feed.

In order to deplete cholesterol, the inventors mixed milk fat with β-cyclodextrin. The cholesterol content was reduced by about 90%.

The hamsters were given for 5 weeks a first regimen having 12.5% of lipids (further characterized on Table 1 below).

TABLE 1
12.5% lipid diet

| | G1 | G2 | G3 | G4 | G5 | G6 | G7 |
|---|---|---|---|---|---|---|---|
| Dry food (100 g) | | | | | | | |
| starch (g) | 37.50 | 37.50 | 37.50 | 37.50 | 37.50 | 37.50 | 37.50 |
| sucrose (g) | 17.50 | 17.50 | 17.50 | 17.50 | 17.50 | 17.50 | 17.50 |
| casein (g) | 20.00 | 20.00 | 20.00 | 20.00 | 20.00 | 20.00 | 20.00 |
| total lipid(g) | 12.50 | 12.50 | 12.50 | 12.50 | 12.50 | 12.50 | 12.50 |
| saturated (g) | 8.59 | 8.55 | 7.80 | 7.60 | 6.94 | 7.55 | 6.92 |
| LA (g) | 0.20 | 0.20 | 0.32 | 0.39 | 0.58 | 0.40 | 0.58 |
| ALA (g) | 0.08 | 0.08 | 0.10 | 0.37 | 0.95 | 0.36 | 0.96 |
| LA/ALA | 2.42 | 2.52 | 3.09 | 1.07 | 0.61 | 1.11 | 0.60 |
| cholesterol (mg) | 34.8 | 3.1 | 32.0 | 31.9 | 27.8 | 3.8 | 3.3 |
| cellulose (g) | 5.00 | 5.00 | 5.00 | 5.00 | 5.00 | 5.00 | 5.00 |
| minerals (g) | 5.00 | 5.00 | 5.00 | 5.00 | 5.00 | 5.00 | 5.00 |
| vitamins (g) | 2.50 | 2.50 | 2.50 | 2.50 | 2.50 | 2.50 | 2.50 |
| Total energy | | | | | | | |
| intake (%) | | | | | | | |
| sugar | 53.30 | 53.30 | 53.30 | 53.30 | 53.30 | 53.30 | 53.30 |
| protein | 19.40 | 19.40 | 19.40 | 19.40 | 19.40 | 19.40 | 19.40 |
| total lipid(g) | 27.30 | 27.30 | 27.30 | 27.30 | 27.30 | 27.30 | 27.30 |
| saturated | 18.75 | 18.66 | 17.03 | 16.57 | 15.15 | 16.47 | 15.09 |
| LA | 0.43 | 0.43 | 0.69 | 0.85 | 1.25 | 0.87 | 1.26 |
| ALA | 0.18 | 0.17 | 0.22 | 0.80 | 2.07 | 0.78 | 2.08 |

After 5 weeks, blood samples were collected by cardiac puncture on starved animals.

Thereafter, the hamsters were given for 12 weeks a regimen having 22% of lipids (further characterized on Table 2 below).

TABLE 2
22% lipid diet

| | G1 | G2 | G3 | G4 | G5 | G6 | G7 |
|---|---|---|---|---|---|---|---|
| Dry food (100 g) | | | | | | | |
| starch (g) | 37.50 | 37.50 | 37.50 | 37.50 | 37.50 | 37.50 | 37.50 |
| sucrose (g) | 8.00 | 8.00 | 8.00 | 8.00 | 8.00 | 8.00 | 8.00 |
| casein (g) | 20.00 | 20.00 | 20.00 | 20.00 | 20.00 | 20.00 | 20.00 |
| total lipid(g) | 22.00 | 22.00 | 22.00 | 22.00 | 22.00 | 22.00 | 22.00 |
| saturated (g) | 15.12 | 15.05 | 13.73 | 13.37 | 12.22 | 13.29 | 12.17 |
| LA (g) | 0.35 | 0.35 | 0.56 | 0.69 | 1.01 | 0.70 | 1.02 |
| ALA (g) | 0.14 | 0.14 | 0.18 | 0.64 | 1.67 | 0.63 | 1.68 |
| LA/ALA | 2.42 | 2.52 | 3.09 | 1.07 | 0.61 | 1.11 | 0.60 |
| cholesterol (mg) | 61.2 | 5.5 | 56.3 | 56.1 | 48.8 | 6.6 | 5.7 |
| cellulose (g) | 5.00 | 5.00 | 5.00 | 5.00 | 5.00 | 5.00 | 5.00 |
| minerals (g) | 5.00 | 5.00 | 5.00 | 5.00 | 5.00 | 5.00 | 5.00 |
| vitamins (g) | 2.50 | 2.50 | 2.50 | 2.50 | 2.50 | 2.50 | 2.50 |
| Total energy | | | | | | | |
| intake (%) | | | | | | | |
| sugar | 39.60 | 39.60 | 39.60 | 39.60 | 39.60 | 39.60 | 39.60 |
| protein | 17.40 | 17.40 | 17.40 | 17.40 | 17.40 | 17.40 | 17.40 |
| total lipid(g) | 43.00 | 43.00 | 43.00 | 43.00 | 43.00 | 43.00 | 43.00 |
| saturated | 29.59 | 29.45 | 26.87 | 26.15 | 23.91 | 25.99 | 23.82 |
| LA | 0.68 | 0.68 | 1.09 | 1.35 | 1.98 | 1.37 | 1.99 |
| ALA | 0.28 | 0.27 | 0.35 | 1.26 | 3.26 | 1.24 | 3.29 |

Note:
in Tables 1 and 2, minerals and vitamins are provided in a mix. Standard mixes provide the recommended daily allowance of minerals and vitamins. LA stands for linoleic acid (ω6 essential fatty acid).

Note: in Tables 1 and 2, minerals and vitamins are provided in a mix. Standard mixes provide the recommended daily allowance of minerals and vitamins. LA stands for linoleic acid (ω6 essential fatty acid).

Fifteen weeks after the beginning of the experiment (10 weeks after the 22% lipid regimen was given), a second blood sample was collected by cardiac puncture on starved animals.

Seventeen weeks after the beginning of the experiment (12 weeks after the 22% lipid regimen was given), a third blood sample was collected by cardiac puncture on animals having retained free access to their feed.

The animals were then sacrified in order to analyze their organs.

The plasma parameters are further depicted in Table 3.

TABLE 3
Blood parameters at three times points.

| | | | | | | | |
|---|---|---|---|---|---|---|---|
| G1 | G2 | G3 | G4 | G5 | G6 | G7 | G8 |

Blood samples at week 5

| | | | | | | | | |
|---|---|---|---|---|---|---|---|---|
| Cholesterol (mg/dl) | 290 | 274 | 283 | 286 | 265 | 255 | 241 | 149 |
| Triglycerides (mg/dl) | 255 | 203 | 255 | 238 | 276 | 282 | 233 | 132 |
| Phospholipids (mg/dl) | 420 | 419 | 438 | 437 | 421 | 410 | 394 | 256 |
| HDL Cholesterol (mg/dl) | 174 | 186 | 180 | 189 | 148 | 157 | 151 | 78 |
| Non-HDL Cholesterol (mg/dl) | 116 | 90 | 103 | 97 | 117 | 99 | 90 | 69 |
| Non-HDL/HDL ratio | 0.80 | 0.49 | 0.58 | 0.54 | 0.83 | 0.66 | 0.60 | 0.89 |

Blood sample at week 15

| | | | | | | | | |
|---|---|---|---|---|---|---|---|---|
| Cholesterol (mg/dl) | 285 | 224 | 283 | 254 | 225 | 212 | 162 | 133 |
| Triglycerides (mg/dl) | 272 | 121 | 260 | 188 | 216 | 174 | 164 | 105 |
| Phospholipids (mg/dl) | 436 | 345 | 435 | 393 | 337 | 329 | 281 | 227 |
| HDL Cholesterol (mg/dl) | 184 | 150 | 182 | 172 | 135 | 131 | 101 | 82 |
| Non-HDL Cholesterol (mg/dl) | 101 | 79 | 101 | 83 | 90 | 81 | 60 | 50 |
| Non-HDL/HDL ratio | 0.56 | 0.52 | 0.58 | 0.62 | 0.68 | 0.78 | 0.74 | 0.64 |

Blood sample at week 17

| | | | | | | | | |
|---|---|---|---|---|---|---|---|---|
| Cholesterol (mg/dl) | 257 | 214 | 256 | 242 | 226 | 208 | 159 | 226 |
| Triglycerides (mg/dl) | 275 | 242 | 252 | 256 | 310 | 219 | 214 | 310 |
| Phospholipids (mg/dl) | 424 | 396 | 419 | 399 | 363 | 376 | 325 | 363 |
| HDL Cholesterol (mg/dl) | 156 | 135 | 162 | 145 | 112 | 117 | 95 | 112 |
| Non-HDL Cholesterol (mg/dl) | 101 | 79 | 93 | 97 | 114 | 91 | 63 | 114 |
| Non-HDL/HDL ratio | 0.69 | 0.61 | 0.61 | 0.68 | 1.08 | 0.82 | 0.70 | 1.08 |

Non-HDL/HDL ratio stands for the atherogenic ratio measured as Non-HDL cholesterol divided by HDL cholesterol values. High atherogenic ratios represent an increased risk of cardiovascular attack.

Besides total cholesterol, cholesterol and saturated fatty acid intakes, the measured blood atherogenic ratio is an independent measure predictive of the risk of cardiovascular diseases.

TABLE 4
animal weight and liver parameters

| | G1 | G2 | G3 | G4 | G5 | G6 | G7 | G8 |
|---|---|---|---|---|---|---|---|---|
| Initial weight (g) | 56.8 | 57.8 | 56.1 | 56.5 | 56.8 | 56.4 | 55.3 | 58.2 |
| Weight at week 17 | 127.0 | 128.0 | 126.0 | 128.7 | 125.0 | 136.0 | 129.5 | 113.3 |
| Difference (g) | 70.3 | 70.3 | 69.9 | 72.0 | 68.3 | 78.7 | 74.3 | 55.2 |
| liver weight (g) | 6.45 | 5.76 | 6.45 | 6.64 | 6.19 | 6.23 | 5.72 | 4.66 |
| SD | 0.81 | 0.69 | 0.55 | 0.78 | 0.44 | 0.53 | 0.69 | 0.42 |
| liver/total %(w:w) | 5.07 | 4.5 | 5.11 | 5.15 | 4.97 | 4.58 | 4.42 | 4.11 |
| Chol (mg/g liver) | 19.39 | 3.98 | 25.96 | 20.13 | 12.65 | 4.47 | 3.03 | 3.55 |
| SD | 8.36 | 2.17 | 8.92 | 7.99 | 7.18 | 2.51 | 1.59 | 1.16 |

As depicted in Table 4, it is clear that the liver weight and its cholesterol content of groups receiving low-cholesterol milk fat regimens (G2 and G7) are close to the values of the control group (G8) or even improved over the control groups G1, G3 and for some aspects of G8.

In addition, the inventors measured plasma values for the fatty acids of all the hamsters in G1 to G7

TABLE 5
Plasma values of fatty acids in groups 1 to 7

| | G1 | G2 | G3 | G4 | G5 | G6 | G7 |
|---|---|---|---|---|---|---|---|
| Triglycerides (% FA) | | | | | | | |
| Saturated | 60.58 | 58.58 | 48.68 | 46.54 | 45.71 | 50.64 | 47.02 |
| Mono unsaturated | 35.77 | 37.61 | 47.19 | 47.38 | 44.43 | 43.55 | 42.17 |
| poly unsaturated | 3.65 | 3.81 | 4.12 | 6.08 | 9.86 | 5.82 | 10.80 |
| EPA + DHA | 0.16 | 0.21 | 0.10 | 0.11 | 0.17 | 0.21 | 0.19 |
| Phospholipids (% FA) | | | | | | | |
| Saturated | 45.47 | 45.31 | 45.79 | 48.61 | 44.78 | 45.41 | 45.28 |
| Mono unsaturated | 36.04 | 32.51 | 33.15 | 30.66 | 27.22 | 27.58 | 22.95 |
| poly unsaturated | 18.49 | 22.18 | 21.05 | 20.73 | 28.00 | 27.01 | 31.77 |
| EPA + DHA | 2.92 | 4.36 | 2.33 | 1.58 | 2.83 | 3.65 | 3.96 |

As demonstrated in Table 5, the reduction of cholesterol in the fat given to the animals results into an increased poly-unsaturated fatty acids content in the plasma, at the expense of the mono-unsaturated fatty acid content for fatty acids in the form of phospholipids, and at the expense of both saturated and mono-unsaturated fatty acids for fatty acids in the form of triglycerides.

The addition of ALA in the regimen results into an increase of plasma poly-unsaturated fatty acids, but EPA and DHA values are not comparatively increased, and even failed to achieve bigger values as obtained in G2.

1. A cholesterol-reduced ruminant dairy product for use as a medicament, said dairy product comprising a cholesterol content between about 10 mg/100 g fat and about 150 mg/100 g fat.

2. The cholesterol-reduced ruminant dairy product of claim 1, wherein the cholesterol content is between about 30 mg/100 g fat and about 150 mg/100 g fat.

3. The cholesterol-reduced ruminant dairy product of claim 1, wherein the cholesterol content is between about 30 mg/100 fat and about 90 mg/100 g fat.

4. The cholesterol-reduced ruminant dairy product according to claim 1 comprising from about 60% to about 70% of saturated fat by weight of total fat.

5. The cholesterol-reduced ruminant dairy product according to claim 1, which is selected from the group consisting of milk, anhydrous milk fat, spread, ice cream, milk cream, cheese, fermented milk, flavored milk and cream.

6. The cholesterol-reduced ruminant dairy product of claim 5, wherein the spread is butter or a spreadable butter.

7. A method of treating and/or preventing a cardiovascular disease in a mammal which comprises the step of administering the dairy product of claim 1 to said mammal.

8. A method of treating and/or preventing an inflammatory disease in a mammal, the method comprising a step of administering the dairy product of claim 1 to said mammal.

9. A method of treating and/or preventing a neurological disease in a mammal which comprises a step of administering the dairy product of claim 1 to said mammal.

10. A method of treating and/or preventing the treatment and/or a liver disease in a mammal which comprises a step of administering the dairy product of claim 1 to said mammal.

11. A method of claim 10, wherein the liver disease is steatosis.

12. A method to decrease the atherogenic ratio in a mammal which comprises a step of administering the dairy product of claim 1 to said mammal.

13. A method to increase poly-unsaturated fatty acids content in a mammal blood which comprises the step of administering the dairy product of claim 1 to said mammal.

14. The method of claim 13, wherein the dairy product long chain poly-unsaturated fatty acids are ω3 fatty acids, which arc selected from the group consisting of Eicosapentaenoic acid (EPA) and Docosahexaenoic acid (DHA).

15. A method to decrease the triglyceride content in a mammal blood which comprises the step of administering the dairy product of claim 1 to said mammal.

16. A method of treating and/or preventing a disease selected from the group consisting of a cardiovascular disease, an inflammatory disease, a liver disease and/or a neurological disease by decreasing atherogenic ratio in a mammal blood by at least 10%, by increasing poly-unsaturated fatty acids content in the mammal blood by decreasing the triglyceride content in the mammal blood through the step of administering the dairy product of claim 1 to said mammal.

17. A cholesterol-reduced ruminant dairy product supplemented with an ω3 fatty acid source selected from the group consisting of α-linolenic acid (ALA), Eicosapentaenoic acid (EPA) and Docosahexaenoic acid (DHA), wherein the dairy product comprises a cholesterol content is between 10 mg/100 g fat and 150 mg/100 g fat.

18. The cholesterol-reduced ruminant dairy product of claim 17 having a molar ratio of ω3 fatty acids to linoleic acid of about 0.2 to about 1.5.

19. The cholesterol-reduced ruminant dairy product of claim 17 having a molar ratio of ω3 fatty acids to linoleic acid of about 1 to about 1.5.

20. A method to increase the content in a mammal blood of long chain poly-unsaturated (ω3) fatty acid comprising administering to the mammal a ruminant dairy product for consumption by the mammal, wherein the ruminant dairy product comprises cholesterol content is between about 10 mg/100 g fat and about 150 mg/g fat.

21. A method to reduce triglyceride content in a mammal blood comprising administering to the mammal a ruminant dairy product for consumption by the mammal, wherein the ruminant dairy product comprises cholesterol content is between about 10 mg/100 g fat and about 150 mg/100 g fat.

22. A method for decreasing blood atherogenic ratio by at least about 10% in a mammal, comprising administering to the mammal a ruminant dairy product, for consumption by the mammal, wherein the ruminant dairy product comprises cholesterol content between about 10 mg/100 g fat and about 150 mg/100 g fat.
